(19) United States
(12) Patent Application Publication
(10) Pub. No.: US 2018/0325785 A1
(43) Pub. Date: Nov. 15, 2018

(54) METHOD FOR MANUFACTURING CAPSULE

(71) Applicant: ASANUMA CORPORATION, Tokyo (JP)

(72) Inventor: Masaaki Ishiwatari, Tokyo (JP)

(21) Appl. No.: 15/774,228

(22) PCT Filed: Feb. 26, 2016
(86) PCT No.: PCT/JP2016/001051

§ 371 Date: May 7, 2018

(30) Foreign Application Priority Data
Nov. 25, 2015 (JP) 2015-229771

(51) Int. Cl. A61K 8/11; A61K 8/06; A61K 8/73

(52) U.S. Cl. CPC A61K 8/11; A61K 8/062; A23P 10/30; A61K 2800/412; A61K 8/731

(57) ABSTRACT

A method for manufacturing a capsule, said method comprising: preparing a mixture by mixing an aqueous solution of a hydrophilic high-molecular gelling agent, said aqueous solution containing at least one member selected from the group consisting of carrageenan, agar, sodium alginate and gellan gum, with an amphiphilic substance compatible with oil and water; adding a cation to the mixture; and removing the amphiphilic substance therefrom.

### CROSS REFERENCE TO RELATED APPLICATIONS

This application is a national stage application, filed under 35 U.S.C. § 371, of International Application No. PCT/JP2016/001051, filed Feb. 26, 2016, which International Application further claims the benefit of and priority to Japanese Application No. 2015-229771, filed Nov. 25, 2015, the contents of both which as are hereby incorporated by reference in their entirety.

### BACKGROUND

### Technical Field

The present invention relates to a method for manufacturing a capsule.

### Description of Related Art

Capsules have been widely used in the fields of, for example, cosmetics, medical products, and foods. Encapsulating a functional substance having a predetermined function enables improvement of the stability and other properties of the functional substrate.

For example, a method for manufacturing such capsules has been proposed.

According to this method, an O/W emulsion is prepared from encapsulated oil droplets (an oil phase) and a water phase containing a capsulizing agent; the prepared O/W emulsion is then dispersed and emulsified in an outer oil phase to prepare an O/W/O emulsion; and thereafter, the water phase is solidified to form capsules.

More specifically, for example, a method for manufacturing microcapsules has been disclosed. This method includes: a step of preparing an O/W emulsion from an inner oil phase and a water phase in which agar or carrageenan functioning as a hydrophilic polymeric gelling agent has been previously dissolved with heat, wherein this preparation of the O/W emulsion is carried out at a temperature equal to or higher than the solidification temperature of the gelling agent; a step of preparing an O/W/O emulsion by dispersing and emulsifying the O/W emulsion in an outer oil phase at a temperature equal to or lower than the solidification temperature of the gelling agent; and a step of forming a capsule by cooling the O/W/O emulsion to a temperature equal to or lower than the solidification temperature of the gelling agent to solidify the water phase. It is described that this method can produce a microcapsule which includes encapsulated micro oil droplets, is highly stable, and can suitably maintain the encapsulated oil droplets after having been applied (see, e.g., Japanese Patent No. 4637993).

Another method for manufacturing capsules has been disclosed. According to this method, a two-fluid nozzle sprays an emulsion liquid containing coenzyme Q10 from the top of a cylindrical coagulation chamber while spaying and mixing a calcium chloride aqueous solution with air, and thereafter, the emulsion liquid containing coenzyme Q10 is gelatinized and brought into a particle state. The emulsion liquid is then collected as an aqueous suspension, and the collected suspension is dehydrated and dried by a common method, thereby manufacturing capsules (see, e.g., International Publication No. 2007/125915).

### BRIEF SUMMARY

However, according to the manufacturing method described in Japanese Patent No. 4637993, in which the O/W/O emulsion is prepared through dispersion and emulsification of the O/W emulsion in the outer oil phase, the outer oil phase remains on the surfaces of the manufactured capsules when the capsules are used. Removal of the remaining outer oil phase needs significant efforts and costs. Thus, the capsules manufactured by the method of Japanese Patent No. 4637993 cannot be used in water-based products such as a skin toner.

It is also difficult to control the particle diameter of the capsules of Japanese Patent No. 4637993. In particular, manufacturing capsules having substantially the same particle diameter of 0.2 mm or more involves difficulty.

On the other hand, implementation of the method of International Publication No. 2007/125915 requires special devices such as the two-fluid nozzle, as previously described. Thus, the method includes complicated manufacturing steps and incurs an increased cost.

In view of the foregoing problems, it is therefore an object of the present invention to provide an inexpensive and simple method for manufacturing a capsule which can be used in water-based products such as a skin toner.

To achieve the object described above, the present invention provides a method for manufacturing a capsule. The method at least includes: preparing a mixture by mixing an aqueous solution of a hydrophilic polymeric gelling agent which is anionic and includes at least one selected from the group consisting of carrageenan, agar, sodium alginate, and gellan gum, with an amphiphilic substance compatible with an oil component and water; adding a cation to the mixture; and removing the amphiphilic substance.

The present invention provides a capsule which is usable in water-based products such as s skin toner can be provided in an inexpensive and simple manner. Further, the present invention provides capsules having substantially the same particle diameter of 0.2 mm or more.

### BRIEF DESCRIPTION OF THE FIGURES

FIG. 1 is a cross-sectional view of a capsule according to an embodiment of the present invention.

### DETAILED DESCRIPTION OF VARIOUS EMBODIMENTS

A favorable embodiment of the present invention will be described below.

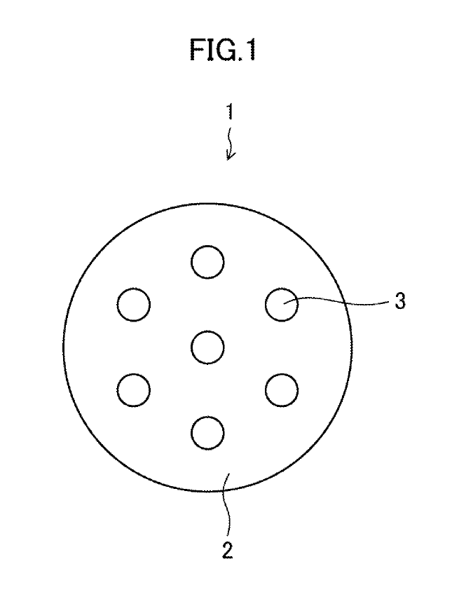

FIG. 1 conceptually shows a capsule of the present invention. As shown in FIG. 1, a capsule 1 of the present invention includes a hydrophilic polymeric gelling agent 2 which is anionic, and encapsulated oil droplets 3 dispersed in the hydrophilic polymeric gelling agent 2.

<Hydrophilic Polymeric Gelling Agent>

In the present invention, carrageenan can be advantageously used as the hydrophilic polymeric gelling agent 2. Carrageenan is a polysaccharide extracted from red algae and can be used as the hydrophilic polymeric gelling agent 2. Examples of the carrageenan usable in the present invention include iota carrageenan which gels upon reacting with calcium ions, which are counter ions.

Note that kappa carrageenan or lambda carrageenan may be used as the carrageenan.

Alternatively, for example, agar, sodium alginate, or gellan gum may be used as the hydrophilic polymeric gelling agent 2, instead of the carrageenan described above. Each of these substances may be used alone or in combination with one or more of the substances.

The concentration of the hydrophilic polymeric gelling agent 2 with respect to the entire aqueous solution of the hydrophilic polymeric gelling agent is preferably from 0.2% to 5% by mass, and more preferably from 0.5% to 4% by mass.

<Encapsulated Oil Droplets>

The encapsulated oil droplets 3 are not limited to any particular substance, and may be comprised of any substance commonly used in cosmetic products and other similar products. Such a substance may be blended at any ratio as long as the advantages of the present invention are ensured. Note that the encapsulated oil droplets of the present invention may be omitted.

A substance for the encapsulated oil droplets may be selected irrespectively of the origin and form. Specifically, an oil for the encapsulated oil droplets may be derived from a plant, an animal, or synthesized, and may be in a solid, semi-solid, or liquid form. Examples of the substances usable as the encapsulated oil droplets include: hydrocarbons, fats and oils, waxes, hardened oils, ester oils, fatty acids, higher alcohols, silicone oils, fluorine-based oils, and oil-based gelling agents.

More specific examples thereof include: hydrocarbons such as an ethylene propylene copolymer, a polyethylene wax, ceresin, a paraffin wax, a microcrystalline wax, a hydrogenated microcrystalline wax, liquid paraffin, squalane, vaseline, and polybutene; fats and oils such as olive oil, castor oil, jojoba oil, and macadamia nut oil; waxes such as beeswax, carnauba wax, candelilla wax, and Japan wax; esters such as triethylhexanoin, isopropyl myristate, isopropyl palmitate, octyldodecyl myristate, glyceryl trioctanoate, sorbitan sesquiisostearate, octyldodecyl isostearate, polyglyceryl diisostearate, diglyceryl triisostearate, glyceryl tribehenate, neopentyl glycol dioctanoate, cholesteryl esters of fatty acid, and N-lauroyl-L-glutamic acid di(cholesteryl beheny octyldodecyl); fatty acids such as oleic acid, palmitic acid, myristic acid, stearic acid, and isostearic acid; higher alcohols such as stearyl alcohol, cetyl alcohol, lauryl alcohol, oleyl alcohol, isostearyl alcohol, and behenyl alcohol; silicones such as a dimethyl polysiloxane having a low polymerization degree, a dimethyl polysiloxane having a high polymerization degree, methylphenyl polysiloxane, a polyether-modified polysiloxane, a copolymer of polyoxyalkylene, alkylmethyl polysiloxane, and methyl polysiloxane copolymer, a cross-linked organopolysiloxane, and a fluorine-modified polysiloxane; fluorine-based oils such as perfluorodecane, perfluorooctane, and perfluoropolyether; and oil-based gelling agents such as dextrin esters of fatty acids, sucrose esters of fatty acids, starch esters of fatty acids, aluminium isostearate, and calcium stearate. Each of these substances may be used alone or in combination with one or more of the substances.

A method for manufacturing a capsule according to an embodiment of the present invention will be described next.

(Where No Oil or Fat is Encapsulated)

<Step of Preparing Aqueous Solution of Hydrophilic Polymeric Gelling Agent>

First, a hydrophilic polymeric gelling agent 2 such as carrageenan or agar is added to ion-exchanged water. The ion-exchanged water is heated to a predetermined temperature (e.g., 90° C.) to dissolve the hydrophilic polymeric gelling agent 2, thereby preparing an aqueous solution containing the hydrophilic polymeric gelling agent 2 dissolved therein.

In this step, in addition to the hydrophilic polymeric gelling agent 2, a commonly-used component (e.g., a moisturizer such as 1,3-butylene glycol and glycerin, an antiseptic such as methylparaben, a colorant such as white pearl powder and gold pearl powder, a granular adjuvant component such as poly-γ-sodium glutamate and hydroxyethyl cellulose) may be blended at any ratio as long as the advantages of the present invention are ensured.

<Step of Mixing Amphiphilic Substance>

Next, an amphiphilic substance is added to the aqueous solution of the hydrophilic polymeric gelling agent described above. Stirring and mixing are carried out using, for example, a propeller stirrer. In this manner, a mixture of the aqueous solution of the hydrophilic polymeric gelling agent and the amphiphilic substance is obtained.

At this time, in the mixture, the hydrophilic polymeric gelling agent 2 is converted into particles (capsules) and dispersed in the amphiphilic substance.

(Where an Oil or Fat is Encapsulated)

First, an aqueous solution containing the hydrophilic polymeric gelling agent 2 dissolved therein is prepared according to the above-described step of preparing the aqueous solution of the hydrophilic polymeric gelling agent.

<Step of Encapsulating Oil Droplets>

Next, the oil droplets are encapsulated. For example, PEG-60 hydrogenated castor oil functioning as a hydrophilic surfactant is added to 1,3-butylene glycol functioning as a moisturizer, heated to a predetermined temperature (e.g., 50° C.) to be dissolved. Next, ion-exchanged water is added, and the resultant mixture is stirred and dissolved. The mixture is then cooled to a predetermined temperature (e.g., 30° C.), thereby obtaining a water phase. An oil (e.g., triethylhexanoin or dimethylpolysiloxane) is added to the water phase. The water phase is processed with a homomixer (emulsifier device), thereby obtaining encapsulated oil droplets 3 as an emulsified liquid phase.

Alternatively, PEG-60 hydrogenated castor oil is added to 1,3-butylene glycol, heated to a predetermined temperature (e.g., 50° C.) to be dissolved. Next, glycerin functioning as a moisturizer, sorbitan sesquiisostearate functioning as a hydrophilic surfactant, and an oil (e.g., triethylhexanoin) are added and dissolved by stirring. The resultant mixed phase is added to ion-exchanged water while stirring is performed. In this manner, the encapsulated oil droplets 3 are obtained as an emulsified liquid phase.

<Step of Preparing O/W Emulsion>

Next, the encapsulated oil droplets prepared are added to the aqueous solution of the hydrophilic polymeric gelling agent. Stirring and mixing are carried out, thereby obtaining an O/W emulsion.

<Step of Mixing Amphiphilic Substance>

Next, an amphiphilic substance is added to the O/W emulsion described above. Stirring and mixing are carried out using, for example, a propeller stirrer. In this manner a mixture of the O/W emulsion and the amphiphilic substance is obtained.

At this time, in the mixture, the hydrophilic polymeric gelling agent 2 is converted into particles (capsules) and dispersed in the amphiphilic substance.

In the present invention, it is also possible that an O/W emulsion or an aqueous solution of the hydrophilic polymeric gelling agent is added to an amphiphilic substance, and stirring and mixing are carried out.

The amphiphilic substance used herein means “a substance compatible with an oil content and water,” and examples thereof include bis-ethoxydiglycol succinate, bis-ethoxydiglycol cyclohexane-1,4-dicarboxylate, diethoxyethyl succinate, 1,2-hexanediol, hexylene glycol, PEG/PPG/polybutylene glycol-8/5/3 glycerin, and PPG-9 diglyceryl ether. Alternatively, it is possible to use, as the amphiphilic substance, a dimethyl ether of a copolymer of polyethylene glycol and polypropylene glycol, such as polyoxyethylene (17) polyoxypropylene (4) dimethyl ether and polyoxyethylene (14) polyoxypropylene (7) dimethyl ether. Each of these substances may be used alone or in combination with one or more of the substances.

To manufacture the capsules 1 having a substantially spherical or ellipsoidal shape, it is preferable to use bis-ethoxydiglycol succinate or bis-ethoxydiglycol cyclohexane-1,4-dicarboxylate. To manufacture the capsules 1 having a fiber-like shape, it is preferable to use a dispersion solvent such as diethoxyethyl succinate, 1,2-hexanediol, or hexylene glycol.

As can be seen, the present invention makes it possible to control the shape of the capsule 1 through selection of the amphiphilic substance to be used.

Since the hydrophilic polymeric gelling agent is dispersed in the amphiphilic substance, the present invention also makes it possible to manufacture capsules having substantially the same particle diameter of 0.2 mm or more.

The added amphiphilic substance can be reused after being subjected to a filtration process. This contributes to cost reduction.

<Step of Adding Cation>

The mixture of the O/W emulsion and the amphiphilic substance (or the mixture of the aqueous solution of the hydrophilic polymeric gelling agent and the amphiphilic substance) obtained in the step of mixing the amphiphilic substance is cooled to a predetermined temperature (e.g., 45° C.). Thereafter, a cation (an inorganic cation or an organic cation) is added to the mixture. Consequently, the hydrophilic polymeric gelling agent 2 comprised of an anionic polymer reacts with the cation, producing a reactant of the hydrophilic polymeric gelling agent 2 and the cation (hereinafter referred to as “the cation reactant”). As a result, the capsule shown in FIG. 1 can be obtained.

It is suitable to use, as the cation for the present invention, any inorganic or organic cation which electrically neutralizes the anionic portion of the hydrophilic polymeric gelling agent. More specifically, examples of the inorganic cation usable in the present invention include calcium ions generated upon dissolution of calcium chloride dihydrate in ion-exchanged water, and magnesium ions generated upon dissolution of magnesium sulfate in ion-exchanged water.

Examples of the organic cation usable in the present invention include a quaternary ammonium cation generated upon dissolution of benzalkonium chloride or cetrimonium chloride in ion-exchanged water.

When the cation described above is added, the hydrophilic polymeric gelling agent comprised of an anionic polymer reacts with the cation, and the hydrophilic polymeric gelling agent gels to form an outer coating. As a result, the capsule shown in FIG. 1 is manufactured.

<Step of Removing Amphiphilic Substance>

According to the present invention, since the hydrophilic polymeric gelling agent 2 (or the O/W emulsion) is dispersed in the amphiphilic substance, the amphiphilic substance may remain on the surface of the capsule 1 when the capsule 1 is used. The amphiphilic substance, which is soluble in water, can be easily removed by filtration and water-washing. Thus, the present invention makes it possible to manufacture the capsule 1 which is usable in water-based products such as a skin toner in an inexpensive and simple manner.

Further, according to present invention, it is possible to make the manufactured capsule 1 resistant to collapse on skin by allowing carrageenan, sodium alginate, and gellan gum to react with calcium ions. On the other hand, it is possible to make the manufactured capsule 1 easy to collapse on skin by allowing agar to react with calcium ions. Thus, the present invention makes it possible to freely deign the collapsibility of the capsule 1. Thus, the present invention can provide both capsules resistant to collapse on skin and capsules easy to collapse on skin.

Furthermore, the present invention makes it possible to encapsulate the oil droplets 3 within the capsule 1, irrespective of the type of the emulsification technique employed.

As can be seen from the foregoing, unlike the known art, the present invention, does not require any special devices, but simply needs to use a simple device (a propeller stirrer) to stir and mix the hydrophilic polymeric gelling agent (or the O/W emulsion) and the amphiphilic substance together. Thus, the present invention makes it possible to manufacture a capsule in an inexpensive and simple manner.

### EXAMPLES

The present invention will be described below with reference to examples. Note that the present invention is not limited to the following examples. Variations and modifications can be made to the following examples on the basis of the spirit of the present invention, and such variations and modifications should not be excluded from the scope of the present invention.

### Examples 1 to 7 and Comparative Examples 1 to 3

<Method for Producing Capsule>

The capsules of Examples 1 to 7 and Comparative Examples 1 to 3 of which the compositions (% by mass) are shown in Table 1 were produced by the following producing method.

TABLE 1

| | | | | | | | | | Compar- | Compar- | Compar- |
|---|---|---|---|---|---|---|---|---|---|---|---|
| | | Exam- | Exam- | Exam- | Exam- | Exam- | Exam- | Exam- | ative | ative | ative |
| | | ple 1 | ple 2 | ple 3 | ple 4 | ple 5 | ple 6 | ple 7 | Example 1 | Example 2 | Example 3 |
| Aqueous | Ion-exchanged water | 38.4 | 38.4 | 38.4 | 39.4 | 39.4 | 26.4 | 37.4 | 39.4 | 38.4 | 41.4 |
| solution of | 1,3-butylene glycol | 2 | 2 | 2 | 2 | 2 | 2 | 2 | 2 | 2 | 2 |
| hydrophilic | Glycerin | 7 | 7 | 7 | 7 | 7 | 7 | 7 | 7 | 7 | 7 |
| polymeric | Methylparaben | 0.1 | 0.1 | 0.1 | 0.1 | 0.1 | 0.1 | 0.1 | 0.1 | 0.1 | 0.1 |
| gelling | Carboxymethylcellulose | — | — | — | — | — | — | — | 1 | — | — |
| agent | Poly-γ-sodium glutamate | — | — | — | — | — | — | — | — | 2 | — |
| | Sodium alginate | — | — | — | 1 | — | — | — | — | — | — |
| | Carrageenan | 2 | 2 | — | — | — | 2 | 2 | — | — | 2 |
| | Agar | — | — | 2 | — | | — | — | — | — | — |
| | Gellan gum | — | — | — | — | 1 | — | — | — | — | — |
| Amphiphilic | Bis-ethoxydiglycol | 47.5 | 47.5 | 47.5 | 47.5 | 47.5 | 47.5 | 47.5 | 47.5 | 47.5 | 47.5 |
| substance | succinate | | | | | | | | | | |
| Aqueous | Ion-exchanged water | 2.7 | 2.7 | 2.7 | 2.8 | 2.85 | 10 | 3 | 2.7 | 2.7 | — |
| solution of | Calcium choride | 0.3 | 0.3 | 0.3 | 0.2 | 0.15 | 5 | 1 | 0.3 | 0.3 | — |
| cation | dihydrate | | | | | | | | | | |
| Total | | 100 | 100 | 100 | 100 | 100 | 100 | 100 | 100 | 100 | 100 |
| Evaluation | Capsule Shape or State | Sphere | Sphere | Sphere | Sphere | Cube | Sphere | Sphere | Solidified | Not formed | Sphere |
| | Capsule Diameter (mm) | 0.3-0.4 | 0.4-0.6 | 0.2-0.4 | 0.5-0.7 | 1-3 | 0.3-0.4 | 0.3-0.4 | — | — | 0.4-0.5 |
| | Stability of Capsule | ∘ | ∘ | ∘ | ∘ | ∘ | ∘ | ∘ | — | — | x |
| | (50° C. 1 month) | | | | | | | | | | |
| | Residual Dispersion | ∘ | ∘ | ∘ | ∘ | ∘ | ∘ | ∘ | ∘ | ∘ | ∘ |
| | Solvent | | | | | | | | | | |

<Preparation of Aqueous Solution of Hydrophilic Polymeric Gelling Agent>

Methylparaben was added to 1,3-butylene glycol, and heated to 50° C. to be dissolved. Glycerin was then added and dissolved by stirring. Next, the hydrophilic polymer of each of Examples was added, wetted and dispersed by stirring. Next, a dispersion phase of the hydrophilic polymer was added to ion-exchanged water, heated to 90° C., and dissolved by stirring. Thereafter, the resultant mixture was cooled to 50° C., thereby obtaining the aqueous solution of the hydrophilic polymeric gelling agent of each of Examples 1 to 7.

For Comparative Examples 1 to 3, carboxymethylcellulose or poly-γ-sodium glutamate was added as the hydrophilic polymer.

<Mixing of Amphiphilic Substance>

Next, bis-ethoxydiglycol succinate (an amphiphilic substance) heated at 50° C. was added to each aqueous solution of the hydrophilic polymeric gelling agent prepared previously. Stirring and mixing were carried out using a propeller stirrer (manufacturer: AS ONE Corporation, product name: STIRRER, P-1) at a speed of 300 rpm, thereby obtaining an mixture of the aqueous solution of the hydrophilic polymeric gelling agent and the amphiphilic substance, for each of Examples and Comparative Examples.

For Example 2, the aqueous solution of the hydrophilic polymeric gelling agent was added to bis-ethoxydiglycol succinate. Stirring and mixing were carried out using the propeller stirrer, thereby obtaining an mixture of the aqueous solution of the hydrophilic polymeric gelling agent and the amphiphilic substance.

<Addition of Cation>

Next, each mixture of the aqueous solution of the hydrophilic polymeric gelling agent and the amphiphilic substance obtained in the step of mixing the amphiphilic substance was cooled to 45° C. Thereafter, ion-exchanged water containing calcium chloride dihydrate dissolved therein was added to the mixture, thereby producing a dispersion of capsules.

<Step of Removing Amphiphilic Substance>

Next, bis-ethoxydiglycol succinate used as the dispersion solvent was removed by filtration and water-washing, thereby producing capsules.

### Examples 8 to 14 and Comparative Example 4

<Method for Producing Capsule>

The capsules of Examples 8 to 14 and Comparative Example 4 of which the compositions (% by mass) are shown in Table 2 were produced by the following producing method.

TABLE 2

| | | Exam- | Exam- | Exam- | Exam- | Exam- | Exam- | Exam- | Comparative |
|---|---|---|---|---|---|---|---|---|---|
| | | ple 8 | ple 9 | ple 10 | ple 11 | ple 12 | ple 13 | ple 14 | Example 4 |
| Aqueous | Ion-exchanged water | 32.6 | 32.6 | 32.6 | 32.6 | 32.6 | 32.6 | 32.6 | 32.6 |
| solution of | 1,3-butylene glycol | 2 | 2 | 2 | 2 | 2 | 2 | 2 | 2 |
| hydrophilic | Glycerin | 10 | 10 | 10 | 10 | 10 | 10 | 10 | 10 |
| polymeric | PPG-9 diglyceryl ether | 2 | 2 | 2 | 2 | 2 | 2 | 2 | 2 |
| gelling | Methylparaben | 0.1 | 0.1 | 0.1 | 0.1 | 0.1 | 0.1 | 0.1 | 0.1 |
| agent | Carrageenan | 0.8 | 0.8 | 0.8 | 0.8 | 0.8 | 0.8 | 0.8 | 0.8 |
| | Agar | 0.6 | 0.6 | 0.6 | 0.6 | 0.6 | 0.6 | 0.6 | 0.6 |
| | Sodium alginate | 0.2 | 0.2 | 0.2 | 0.2 | 0.2 | 0.2 | 0.2 | 0.2 |
| | Hydroxyethyl cellulose | 0.2 | 0.2 | 0.2 | 0.2 | 0.2 | 0.2 | 0.2 | 0.2 |
| | Gold pearl powder | 1 | 1 | 1 | 1 | 1 | 1 | 1 | 1 |
| Amphiphilic | Bis-ethoxydiglycol | 47.5 | 47.5 | 27.5 | — | — | — | — | — |
| substance | succinate | | | | | | | | |
| | Bis-ethoxydiglycol | — | — | 20 | 35 | — | 22.5 | 30 | — |
| | cyclohexane-1,4- | | | | | | | | |
| | dicarboxylate | | | | | | | | |
| | Diethoxyethyl succinate | — | — | — | — | 37.5 | 10 | 5 | — |
| | 1,2-hexanediol | — | — | — | — | — | 15 | 5 | — |
| | PEG/PPG/polybutylene | — | — | — | 12.5 | 10 | — | — | — |
| | glycol-8/5/3 glycerin | | | | | | | | |
| | PPG-9 diglyceryl ether | — | — | — | — | — | — | 7.5 | — |
| | POE methyl polysiloxane | — | — | — | — | — | — | — | 0.5 |
| | copolymer | | | | | | | | |
| | Dimethyl polysiloxane | — | — | — | — | — | — | — | 47 |
| | (5CPS) | | | | | | | | |
| Aqueous | Ion-exchanged water | 2.7 | 2.7 | 2.7 | 2.7 | 2.7 | 2.7 | 2.7 | 2.7 |
| solution of | Calcium choride | 0.3 | 0.3 | 0.3 | 0.3 | 0.3 | 0.3 | 0.3 | 0.3 |
| cation | dihydrate | | | | | | | | |
| Total | | 100 | 100 | 100 | 100 | 100 | 100 | 100 | 100 |
| | RPM of propeller | 400 rpm | 100 rpm | 400 rpm | 200 rpm | 200 rpm | 200 rpm | 200 rpm | 200 rpm |
| Evaluation | Capsule Shape or State | Sphere | Sphere | Sphere | Sphere | Sphere | Ellipsoid | Sphere | Sphere |
| | Capsule Diameter (mm) | 0.2-0.4 | 1-3 | 0.7-1 | 2-3 | 2-3 | 1-3 | 0.5-1 | 0.3-0.4 |
| | Stability of Capsule | ∘ | ∘ | ∘ | ∘ | ∘ | ∘ | ∘ | — |
| | (50° C. 1 month) | | | | | | | | |
| | Residual Dispersion | ∘ | ∘ | ∘ | ∘ | ∘ | ∘ | ∘ | x |
| | Solvent | | | | | | | | |

<Aqueous Solution of Hydrophilic Polymeric Gelling Agent>

Methylparaben was added to 1,3-butylene glycol, and heated to 50° C. to be dissolved. Glycerin and PPG-9 diglyceryl ether were then added and dissolved by stirring. Next, hydrophilic polymers and hydroxyethyl cellulose were added, wetted and dispersed by stirring, for each of Examples and Comparative Example. Next, a dispersion phase of the hydrophilic polymers and hydroxyethyl cellulose was added to ion-exchanged water, heated to 90° C., and dissolved by stirring. Thereafter, the resultant mixture was cooled to 50° C., and gold pearl powder was added to the mixture and dispersed by stirring, thereby obtaining an aqueous solution of the hydrophilic polymeric gelling agents, for each of Examples 9 to 15 and Comparative Example 4.

<Mixing of Amphiphilic Substance>

Next, for Examples 9 to 11, 13 and 14, the respective aqueous solution of the hydrophilic polymeric gelling agents was added to the respective amphiphilic substance(s) (bis-ethoxydiglycol succinate, bis-ethoxydiglycol cyclohexane-1,4-dicarboxylate, diethoxyethyl succinate, 1,2-hexanediol, PEG/PPG/polybutylene glycol-8/5/3 glycerin, and/or PPG-9 diglyceryl ether) heated at 50° C. Stirring and mixing were performed using the propeller stirrer at the respective rpm shown in Table 2. In this manner, the respective mixtures of the aqueous solution of the hydrophilic polymeric gelling agents and the amphiphilic substance(s) were obtained.

For Examples 8 and 12, the respective amphiphilic substance(s) (bis-ethoxydiglycolsuccinate, diethoxyethyl succinate, 1,2-hexanediol, and/or PEG/PPG/polybutylene glycol-8/5/3 glycerin) heated at 50° C. was added to the prepared aqueous solution of the hydrophilic polymeric gelling agents. Stirring and mixing were performed using the propeller stirrer at the respective rpm shown in Table 2. In this manner, the respective mixtures of the aqueous solution of the hydrophilic polymeric gelling agents and the amphiphilic substance(s) were obtained.

<Addition of Cation>

Next, each mixture of the aqueous solution of the hydrophilic polymeric gelling agents and the amphiphilic substance(s) obtained in the step of mixing the amphiphilic substance was cooled to 45° C. Thereafter, ion-exchanged water containing calcium chloride dihydrate dissolved therein was added to the mixture, thereby producing a dispersion of capsules.

<Step of Removing Amphiphilic Substance>

Next, bis-ethoxydiglycol succinate, bis-ethoxydiglycol cyclohexane-1,4-dicarboxylate, diethoxyethyl succinate, 1,2-hexanediol, PEG/PPG/polybutylene glycol-8/5/3 glycerin, PPG-9 diglyceryl ether that were used as the dispersion solvent were removed by filtration and water-washing. In this manner, the respective capsules were produced.

### Examples 15 to 20

<Method for Producing Capsule>

The capsules of Examples 15 to 20 of which the compositions (% by mass) are shown in Table 3 were produced by the following producing method.

TABLE 3

| | | Example 15 | Example 16 | Example 17 | Example 18 | Example 19 | Example 20 |
|---|---|---|---|---|---|---|---|
| Aqueous | Ion-exchanged water | 39.2 | 39.2 | 39.2 | 39.2 | 23.6 | 31.9 |
| solution of | 1,3-butylene glycol | 2 | 2 | 2 | 2 | 2 | 2 |
| hydrophilic | Glycerin | 5 | 5 | 5 | 5 | 5 | 5 |
| polymeric | Methylparaben | 0.1 | 0.1 | 0.1 | 0.1 | 0.1 | 0.1 |
| gelling | Carrageenan | 1.5 | 1.5 | 1.5 | 1.5 | 1.5 | 1.5 |
| agent | Agar | 1 | 1 | 1 | 1 | 1 | 1 |
| | Poly-γ-sodium glutamate | 0.1 | 0.1 | 0.1 | 0.1 | 0.1 | 0.1 |
| | Hydroxyethyl cellulose | 0.1 | 0.1 | 0.1 | 0.1 | 0.1 | 0.1 |
| | White pearl powder | 0.3 | 0.3 | 0.3 | 0.3 | 0.3 | 0.3 |
| | Gold pearl powder | 0.2 | 0.2 | 0.2 | 0.2 | 0.2 | 0.2 |
| Amphiphilic | Bis-ethoxydiglycol | 47.5 | 47.5 | 45 | 45 | 47.5 | 47.5 |
| substance | succinate | | | | | | |
| Aqueous | Ion-exchanged water | 2.5 | 2.5 | 5 | 5 | 2.6 | 2.7 |
| solution of | Calcium choride | 0.5 | — | — | — | 0.1 | 0.3 |
| cation | dihydrate | | | | | | |
| | Magnesium sulfate | — | 0.5 | — | — | — | — |
| | Benzalkonium chloride | — | — | 0.5 | — | — | — |
| | Cetrimonium chloride | — | — | | 0.5 | 0.3 | — |
| Encapsulated | Ion-exchanged water | — | — | — | — | 6.4 | 3.8 |
| oil droplets | 1,3-butylene glycol | — | — | — | — | 4 | 1 |
| | Glycerin | — | — | — | — | — | 1.5 |
| | PEG-50 hydrogenated | — | — | — | — | 0.2 | 0.2 |
| | castor oil | | | | | | |
| | Sorbitan sesquisostearate | — | — | — | — | — | 0.4 |
| | Triethylhexanoin | — | — | — | — | 2 | 0.4 |
| | Dimethyl polysiloxane | — | — | — | — | 3 | — |
| | (5CPS) | | | | | | |
| Total | | 100 | 100 | 100 | 100 | 100 | 100 |
| Evaluation | Capsule Shape or State | Sphere | Ellipsoid | Sphere | Sphere | Sphere | Sphere |
| | Capsule Diameter (mm) | 0.2-0.4 | 0.4-0.7 | 0.2-0.3 | 0.2-0.4 | 0.2-0.4 | 0.2-0.3 |
| | Stability of Capsule | ∘ | ∘ | ∘ | ∘ | ∘ | ∘ |
| | (50° C. 1 month) | | | | | | |
| | Residual Dispersion | ∘ | ∘ | ∘ | ∘ | ∘ | ∘ |
| | Solvent | | | | | | |

<Aqueous Solution of Hydrophilic Polymeric Gelling Agent>

Methylparaben was added to 1,3-butylene glycol, and heated to 50° C. to be dissolved. Glycerin was then added and dissolved by stirring. Next, hydrophilic polymers, poly-γ-sodium glutamate, and hydroxyethyl cellulose were added, wetted and dispersed by stirring. Next, a dispersion phase of the hydrophilic polymers, poly-γ-sodium glutamate, and hydroxyethyl cellulose was added to ion-exchanged water, heated to 90° C., and dissolved by stirring. Thereafter, the resultant mixture was cooled to 50° C., and white pearl powder and gold pearl powder were added to the mixture and dispersed by stirring, thereby obtaining an aqueous solution containing the hydrophilic polymeric gelling agents, for each of Examples 16 to 21.

<Encapsulated Oil Phase>

The encapsulated oil phase of Example 19 was produced in the following manner. First, PEG-60 hydrogenated castor oil was added to 1,3-butylene glycol, heated to 50° C. to be dissolved. Next, ion-exchanged water was added. Following stirring and dissolution, the mixture was cooled to 30° C., thereby obtaining a water phase. Thereafter, triethylhexanoin and dimethyl polysiloxane were added to the water phase. The water phase was processed using a homomixer (emulsifier device), thereby obtaining an encapsulated oil phase as an emulsified liquid phase.

The encapsulated oil phase of Example 20 was produced in the following manner. First, PEG-60 hydrogenated castor oil was added to 1,3-butylene glycol, heated to 50° C. to be dissolved. Next, glycerin, sorbitan sesquiisostearate, and triethylhexanoin were added and dissolved by stirring. The resultant mixed phase was added to ion-exchanged water while stirring was performed. In this manner, the encapsulated oil phase was obtained as an emulsified liquid phase.

<O/W Emulsion>

Next, the prepared encapsulated oil phase was added to the respective aqueous solution of the hydrophilic polymeric gelling agents. Following stirring and mixing, an 01W emulsion was obtained.

<Mixing of Amphiphilic Substance>

Next, for each of Examples 15 to 18, bis-ethoxydiglycol succinate (an amphiphilic substance) heated at 50° C. was added to the respective aqueous solution of the hydrophilic polymeric gelling agents prepared previously. Stirring and mixing were carried out using the propeller stirrer at a speed of 400 rpm, thereby obtaining a mixture of the aqueous solution of the hydrophilic polymeric gelling agents and the amphiphilic substance.

For each of Examples 19 and 20, bis-ethoxydiglycol succinate (an amphiphilic substance) heated at 50° C. was added to the respective O/W emulsion prepared previously. Stirring and mixing were carried out using the propeller stirrer at a speed of 400 rpm, thereby obtaining a mixture of the O/W emulsion and the amphiphilic substance.

<Addition of Cation>

Next, the respective mixture of the aqueous solution of the hydrophilic polymeric gelling agents and the amphiphilic substance (for Examples 19 and 20, the respective mixture of the O/W emulsion and the amphiphilic substance) obtained in the step of mixing the amphiphilic substance was cooled to 45° C. Thereafter, ion-exchanged water containing at least one of calcium chloride dihydrate, magnesium sulfate, benzalkonium chloride, or cetrimonium chloride, dissolved therein was added to the mixture, thereby producing a dispersion of capsules.

<Step of Removing Amphiphilic Substance>

Next, bis-ethoxydiglycol succinate used as the dispersion solvent was removed by filtration and water-washing, thereby producing capsules.

### Examples 21 to 24

<Method for Producing Capsule>

The capsules of Examples 21 to 24 of which the compositions (% by mass) are shown in Table 4 were produced by the following producing method.

TABLE 4

| | | Example 21 | Example 22 | Example 23 | Example 24 |
|---|---|---|---|---|---|
| Aqueous | Ion-exchanged water | 44.45 | 44.45 | 37.5 | 42.6 |
| solution of | 1,3-butylene glycol | 3 | 3 | 10 | 2 |
| hydrophlic | Glycerin | — | — | — | 2 |
| polymeric | Methylparaben | 0.15 | 0.15 | 0.1 | 0.1 |
| getting agent | Carrageenan | 1 | 1 | 0.6 | 1 |
| | Agar | 0.4 | 0.4 | 0.4 | 0.5 |
| | Sodium alginate | 0.2 | 0.2 | 0.2 | — |
| | Gellan gum | — | — | 0.4 | 0.5 |
| | Polyvinyl alcohol | — | — | | 0.5 |
| | Gold pearl powder | 0.8 | 0.8 | 0.8 | 0.8 |
| Amphiphlic | Bis-ethoxydiglycol succinate | — | — | — | 37 |
| substance | 1,2-hexanediol | 46 | 34.5 | 47 | 10 |
| | Ion exchanged water | — | 10 | — | — |
| | Common salt | — | 1.5 | — | — |
| Aqueous | Ion-exchanged water | 3 | 3 | 2.5 | 2.5 |
| solution of | Calcium chloride dihydrate | 1 | 1 | 0.5 | 0.5 |
| cation | | | | | |
| Total | | 100 | 100 | 100 | 100 |
| Evaluation | Capsule Shape or State | Long | Fiber-like | Fiber-like | Fiber-like |
| | | fiber-like | shape | shape | shape |
| | | shape | | | |
| | Longitudinal Diameter (mm) of | 10-50 | 5-10 | 5-10 | 3-7 |
| | Capsule | | | | |
| | Short Diameter (mm) of Capsule | 0.3-10 | 0.2-0.5 | 0.1-0.3 | 0.1-0.2 |
| | Stability of Capsule (50° C., 1 month) | ◯ | ◯ | ◯ | ◯ |
| | Residual Dispersion Solvent | ◯ | ◯ | ◯ | ◯ |

<Aqueous Solution of Hydrophilic Polymeric Gelling Agent>

Methylparaben was added to 1,3-butylene glycol, and heated to 50° C. to be dissolved. Glycerin was then added and dissolved by stirring. Next, the respective hydrophilic polymers and polyvinyl alcohol were added, wetted and dispersed by stirring. Next, a dispersion phase of the hydrophilic polymer and polyvinyl alcohol was added to ion-exchanged water, heated to 90° C., and dissolved by stirring. Thereafter, the resultant mixture was cooled to 50° C., and gold pearl powder was added to the mixture and dispersed by stirring, thereby obtaining an aqueous solution containing the hydrophilic polymeric gelling agents, for each of Examples 21 to 24.

<Mixing of Amphiphilic Substance>

Next, an amphiphilic substance(s) (1,2-hexanediol and/or bis-ethoxydiglycol succinate) heated at 50° C. was added to each aqueous solution of the hydrophilic polymeric gelling agents prepared previously. Stirring and mixing were carried out using the propeller stirrer at a speed of 500 rpm, thereby obtaining a mixture of the aqueous solution of the hydrophilic polymeric gelling agents and the amphiphilic substance(s).

For Example 22, in order to facilitate the production of capsules having a fiber-like shape, ion-exchanged water and common salt (substance having salting-out effect) were added to the amphiphilic substances.

<Addition of Cation>

Next, each mixture of the aqueous solution of the hydrophilic polymeric gelling agents and the amphiphilic substance obtained in the step of mixing the amphiphilic substance was cooled to 45° C. Thereafter, ion-exchanged water containing calcium chloride dihydrate dissolved therein was added to the mixture, thereby producing a dispersion of capsules.

<Step of Removing Amphiphilic Substance>

Next, 1,2-hexanediol and/or bis-ethoxydiglycol succinate used as the dispersion solvent, and common salt were removed by filtration and water-washing, thereby producing capsules.

The shape, diameter, stability, and residual solvent of the respective capsules were evaluated in the following manner. The results of the foregoing are shown in Tables 1 to 4.

<Capsule Shape or State>

The shape and state of the capsules of Examples and Comparative Examples were evaluated visually.

<Capsule Diameter>

The diameters of the capsules of Examples and Comparative Examples were visually measured using a ruler.

<Stability of Capsule>

The capsules produced were stored in a threaded test tube at 50° C. for one month. Thereafter, the capsules were visually checked for changes such as deformation, rupture, and aggregation.

Evaluation Criteria

A circle indicates where no or slight deformation, rupture, and aggregation of capsules were observed.

A cross indicates where moderate or obvious deformation, rupture, and/or aggregation of capsules were observed.

<Residual Dispersion Solvent>

It was evaluated whether residual dispersion solvent (i.e. the amphiphilic substance(s) remaining on the surfaces of the capsules) was present or absent, using a liquid chromatograph (manufacture: SHIMADZU CORPORATION, product name: Prominence).

Evaluation Criteria

A circle indicates where the concentration was lower than 100 ppm.

A cross indicates where a oil film or oil floated on water when the capsules were immersed, or the concentration was 100 ppm or more.

As shown in Tables 1 to 3, capsules which are highly stable, have the shape of a sphere, cube, or ellipsoid, and substantially the same particle diameter of 0.2 mm or more can be obtained, according to Examples 1 to 20 where the hydrophilic polymeric gelling agent (at least one of carrageenan, agar, sodium alginate, or gellan gum) that is anionic was mixed with the amphiphilic substance (at least one of bis-ethoxydiglycol succinate, bis-ethoxydiglycol cyclohexane-1,4-dicarboxylate, diethoxyethyl succinate, 1,2-hexanediol, PEG/PPG/polybutylene glycol-8/5/3 glycerin, or PPG-9 diglyceryl ether), and the cation (calcium ions, magnesium ions, or a quaternary ammonium cation) was added to the resultant mixture.

As shown in Table 4, capsules which are highly sable and have a fiber-like shape can be provided, according to Examples 21 to 24 where the hydrophilic polymeric gelling agent (at least one of carrageenan, agar, sodium alginate, or gellan gum) that is anionic was mixed with the amphiphilic substance (at least one of bis-ethoxydiglycol succinate or 1,2-hexanediol), and the cation (calcium ions) was added to the resultant mixture.

By contrast, as shown in Tables 1 and 2, according to Comparative Example 1 where none of carrageenan, agar, sodium alginate, and gellan gum was used, the capsules themselves were solidified (specifically, the hydrophilic polymer was solidified together without being shaped into particles) Similarly, according to Comparative Example 2, no capsules were able to be produced.

The table shows that according to Comparative Example 3 where no cation was added, capsules were produced, but their stability was low. According to Comparative Example 4 where the amphiphilic substances of the present invention were not mixed, oil remained obviously. This means that the capsules of Comparative Example 4 can be blended in oil-based and W/O-based products, but cannot be blended in water-based products.

### INDUSTRIAL APPLICABILITY

As can be seen from the foregoing description, the present invention is particularly useful for manufacturing capsules for use in the fields of, for example, cosmetics, medical products, and foods.

### DESCRIPTION OF REFERENCE CHARACTERS

1 Capsule

2 Anionic and Hydrophilic Polymeric Gelling Agent

3 Encapsulated Oil Droplet

1-5. (canceled)

6. A method for manufacturing a capsule, the method comprising the steps of:
preparing a mixture by mixing an aqueous solution of a hydrophilic polymeric gelling agent which is anionic and includes at least one selected from the group consisting of carrageenan, agar, sodium alginate, and gellan gum, with an amphiphilic substance compatible with an oil component and water;
adding a cation to the mixture; and
removing the amphiphilic substance.

7. The method of claim 6, wherein the amphiphilic substance is at least one substance selected from the group consisting of: bis-ethoxydiglycol succinate, bis-ethoxydiglycol cyclohexane-1,4-dicarboxylate, diethoxyethyl succinate, 1,2-hexanediol, and hexylene glycol.

8. The method of claim 6, wherein the cation is at least one selected from the group consisting of: a calcium ion, a magnesium ion, or a quaternary ammonium cation.

9. The method of claim 6, wherein, in the preparation of the mixture, the amphiphilic substance is added to the aqueous solution of the hydrophilic polymeric gelling agent.

10. The method of claim 6, wherein, in the preparation of the mixture, the aqueous solution of the hydrophilic polymeric gelling agent is added to the amphiphilic substance.
